(19) United States
(12) Patent Application Publication
(10) Pub. No.: US 2026/0115039 A1
(43) Pub. Date: Apr. 30, 2026

(54) Droplet tracker system and method for ophthalmic medicine delivery confirmation

(71) Applicant: Nanodropper, Inc., Rochester, MN (US)

(72) Inventors: Elias Lee Baker, Rochester, MN (US); Mackenzie Andrews, Denver, CO (US); Jake Hannafious, Denver, CO (US); Allisa Jungha Song, Rochester, MN (US)

(21) Appl. No.: 19/371,533

(22) Filed: Oct. 28, 2025

Related U.S. Application Data
(60) Provisional application No. 63/712,651, filed on Oct. 28, 2024.

(51) Int. Cl. A61F 9/00; A61B 5/00; A61B 5/01; A61B 5/11

(52) U.S. Cl. CPC A61F 9/0026; A61B 5/0077; A61B 5/015; A61B 5/1103; A61B 5/4839

(57) ABSTRACT

A system for tracking delivery of a medication to a user’s eye and a method of use. The system comprises at least one camera for capturing one or more images of an eye surface of the user’s eye before, during, and after a time when the medication is applied to the user’s eye. The images captured are visual or thermal images, and for example, a temperature of the eye, eyelid, and periocular surfaces may be captured as well as visualization of the location of the eyelid or iris. If a second camera is used, it may be the same or different type of camera as the first camera and may capture one or more second images of the user’s eye before, during, and after the time when the droplets of the medication are applied to the user’s eye. A processor is provided for comparing the first images captures and/or comparing the first images to the second images, in order to determine when to deploy the droplets and/or if the droplets of the medication were successfully applied to the user’s eye.

### BACKGROUND

The present invention relates to eye medication delivery systems, and more specifically to a system configured to track adherence and/or compliance of a spray of liquid medication to a user’s eye.

One main concern for at-home use of micro-volume misters and spritzers is that the individual droplets in the mist are too fine for the user to feel and determine whether the medication was administered successfully. Additionally, the total volume of a spray/mist is smaller than a drop from a standard dropper bottle. The total volume of a spray/mist may be about 5-20uL, and the total volume of a drop from a standard dropper bottle may be about 35-70uL.

Home delivery of eye drops and topical ophthalmic medications have made it difficult to track adherence and compliance of such products because there is no way to ensure that the eye drops made it onto the eye surface for a proper treatment. Many solutions have been proposed such as tracking whether the cap on an eye drop bottle was opened and whether the eye drop bottle was turned upside down. However, these are indirect measures and fail to determine whether drops actually made it onto the surface of the eye.

### SUMMARY

An aspect of the present invention relates to an adherence tracker for ophthalmic solutions, commonly known as “eye drops”. The adherence tracker is used to determine if ophthalmic solution has been delivered to the eye successfully or in an amount sufficient for the intended treatment and/or to the target location on or near the eye. The adherence tracker utilized one or more cameras and optionally in some embodiments one or more sensors to determine if the ophthalmic solution has been successfully delivered as noted above. The adherence tracker is configured for use with a supply of ophthalmic solution for delivery to a user’s eye. The ophthalmic solution may be provided in a delivery device for delivery of a spray or mist of ophthalmic solution as well as a container for delivery of ophthalmic solution such as an eye drop bottle or the like. The adherence tracker may be provided as a standalone product for use in connection with the delivery of the ophthalmic solution from whatever device the supply of ophthalmic supply is delivered from or provided attached thereto or integrated with a delivery device as a unit.

An aspect of the present disclosure relates to a system for tracking delivery of medication to a user’s eye comprising one or more cameras for capturing one or more images of the user’s eye, before, during, and after delivery of medication to the user’s eye.

In one or more embodiments, the one or more cameras are provided on a delivery device for delivery of the medication to the user’s eye and in operable connection therewith.

In one or more embodiments, the one or more cameras are provided with the delivery device for delivery of the medication to the user’s eye and are configured to track delivery of medication to the user’s eye from the delivery device without being integrated into the delivery device.

The one or more cameras comprise a thermal camera, an optical camera, a multispectral camera, a hyperspectral imaging camera, or a combination thereof and wherein the one or more images comprise one or more optical images, thermal images or combinations thereof.

In one or more embodiments, the system comprises a single infrared camera to capture one or more images of the user’s eye before, during, and after delivery of medication to the user’s eye and a processor configured to compare one or more images captured before, during, or after delivery of medication to the user’s eye to determine if the medication was successfully applied to the user’s eye.

In one or more embodiments, the system comprises an infrared camera and a visible light camera to capture one or more images of the user’s eye before, during, and after delivery of medication to the user’s eye and a processor configured to compare one or more images captured from one or both of the infrared camera and visible light camera before, during, or after delivery of medication to the user’s eye to determine if the medication was successfully applied to the user’s eye.

In one or more embodiments, the one or more cameras are configured for capturing one or more first optical or thermal images of a surface of the user’s eye, before, during, and after delivery of medication.

In one or more embodiments the system further comprises a processor for comparing one or more images captures before, during, and after delivery of medication to the user’s eye to determine if the medication was successfully applied to the user’s eye.

In one or more embodiments, the medication is a liquid formulation. For example, the medication is a liquid suspension or aqueous solution and the delivery device is configured to deliver the medication in the form of a droplet, a mist, or a spray.

In one or more embodiments, the system of claim 1 and further comprising a processor configured to determine an open or close state of an eye lid of the eye, an on or off target time for delivery of medication, an ocular or periocular surface temperature or combination thereof.

In one or more embodiments, the processor utilizes an artificial intelligence algorithm for determination.

Another aspect of the present disclosure relates to a method for tracking delivery of a medication to a user’s eye comprising capturing one or more first images of an eye surface of a user’s eye with one or more cameras, wherein at least one of the first images is taken before, during, and after a time when the medication is delivered to the eye surface; capturing one or more second images of the user’s eye, wherein the one or more second images are taken during the time when the medication is delivered to the user’s eye, wherein the one or more second images are captured with the one or more cameras; and comparing the first one or more images and the one or more second images to determine a timing and/or a location of the user’s eye where the medication should be or was delivered.

In one or more embodiments, the method further comprises providing the one or more cameras on a delivery device for the delivery of the medication to the user’s eye.

In one or more embodiments, the method further comprises capturing one or more first images of the user’s eye surface with a first camera and capturing the one or more second images with a second camera wherein the first camera and second camera comprise a thermal camera, optical camera, or combination thereof.

In one or more embodiments, the method further comprises using the one or more first and one or more second images to determine an open or close state of an eye lid of the eye, an on or off target time for delivery of medication, an ocular or periocular surface temperature or combination thereof and coordinating the information captured by the one or more cameras with the drug delivery device to independently deploy medication between the user’s blinks.

### BRIEF DESCRIPTION OF THE DRAWINGS

FIG. 1 is a perspective view of a spray tracker system according to one or more embodiments described herein.

### DETAILED DESCRIPTION

An adherence tracker for ophthalmic solutions, commonly known as “eye drops” is described herein. The adherence tracker is used to determine if ophthalmic solution has been delivered to the eye successfully or in an amount sufficient for the intended treatment and/or to the target location on or near the eye. The adherence tracker may also be used to determine a time when the ophthalmic solution should be delivered to the user’s eye. The adherence tracker utilizes one or more cameras and optionally in some embodiments, one or more sensors to determine if the ophthalmic solution has been successfully delivered as noted above. The adherence tracker is configured for use with a supply of ophthalmic solution for delivery to a user’s eye. The ophthalmic solution supply may be provided for delivery in a delivery device for delivery of a spray or mist of the ophthalmic solution or in a container for delivery of ophthalmic solution such as an eye drop bottle or the like. The adherence tracker may be provided as a standalone product for use in connection with the delivery of the ophthalmic solution from whatever device the supply of ophthalmic supply is delivered from including a device configured to provide a spray of solution or a device configured to provide a drop of solution. The adherence tracker may alternatively be provided attached to a delivery device for the ophthalmic solution or integrated with a delivery device there and provides as a unit.

In further detail, the adherence tracker is also referred to as a droplet or micro-droplet tracker system for tracking delivery of a medication to user’s eye and is described herein. The system for tracking delivery of medication to a user’s eye comprises one or more cameras for capturing one or more images of the user’s eye, before, during, and after delivery of medication to the user’s eye; and the one or more cameras may be provided on a delivery device for delivery of the medication to the user’s eye and in operable connection therewith is described herein or may be provided on an adherence tracker support which may be used in conjunction with a delivery device of any type for delivery of medication to the user’s eye.

What is meant by medication as used throughout this application is generally a liquid composition commonly referred to as ophthalmic solutions such as eye drops but also includes aqueous compositions and medications delivered in the form of sprays or the like. For example, the systems and method described herein are capable of tracking mediation delivered to the eye generally or locations of the eye specifically as a liquid droplet or droplets of medication or the liquid droplet or droplets may also be delivered in the form of a spray, spray of micro-droplets, aerosol, or the like to a user’s eye(s) as described herein. In one or more embodiments, the system may be configured to track whether the liquid was successfully applied and/or delivered to the surface of the user’s eye(s). What is meant by successfully delivered is that sufficient medication was delivered to the user’s eye or intended target location on or near the eye and/or a sufficient treatment amount was delivered to the user’s eyer per the recommended or prescribed dose.

As described herein, the one or more cameras may comprise a thermal camera, an optical camera, a multispectral camera or otherwise utilize hyperspectral imaging systems or cameras or a combination thereof and the one or more images captured may comprise one or more optical images, thermal images or combinations thereof.

The system described herein comprises one or more cameras and/or one or more sensors or systems to detect a user’s eye and/or a medication applied thereto. The one or more cameras and/or sensors and/or systems may be configured to capture and/or detect a set of parameters that may be used to evaluate whether an on-target and/or off-target spray application occurred. For example, the cameras and/or sensors and/or systems may be configured to capture one or more images, temperatures, and/or colors of one or more frames and/or sections of the eye(s) and/or visible light spectrum images and infrared images thereof. The parameters may be changeable and/or adjustable for different conditions and uses.

The data collected by the cameras and/or sensors may be stored on a storage component and/or processed by a processor. In one or more embodiments, the processor executes one or more algorithm(s) to process the data and/or images. The processor may be coupled to a non-transitory computer-readable medium. The spray tracker system may produce a signal and/or other output to indicate to a user whether an on-target and/or off-target spray was applied.

In one or more embodiments, the system is a spray tracker system comprising an infrared camera. The infrared camera may be used to detect and/or capture a change in eye surface temperature, such as eye corneal surface, eyelid surface, or periorbital skin temperature, when the solution or suspension is applied to the eye. The infrared camera may capture one or more images before, during, and/or after the spray is applied to the eye. The infrared camera may capture one or more images to collect and/or capture the temperature data of the eye surface. The one or more images may be evaluated by a processor to determine a change in temperature and/or whether the change in temperature was significant. A change in the eye corneal surface temperature may indicate that an on-target spray was applied, whereas an off-target spray may result in no change in eye corneal surface temperature and/or an incorrect eye corneal surface temperature change. An incorrect eye corneal surface temperature may be a temperature that does not correlate and/or match a set temperature parameter.

In one or more embodiments, the system is a spray tracker system comprising a single infrared camera to capture one or more images, or a plurality of images of the user’s eye before, during, and after delivery of medication to the user’s eye and a processor configured to read a single image or compare two or more images captured before, during, or after delivery of medication to the user’s eye to determine if the medication was successfully applied to the user’s eye.

In one or more embodiments, the spray tracker system comprises a visible light camera. The visible light camera may detect and/or capture a position of the eye(s), whether an eyelid is open or closed, and/or the borders for defining on- and/or off-target areas. For example, the visible light camera may capture the alignment of the eye(s) with respect to the spray tracker system.

In one or more embodiments, the spray tracker system comprises a multispectral camera or otherwise utilize hyperspectral imaging systems or cameras alone or in combination with other cameras and/or sensors.

In some embodiments, the spray tracker system may comprise both a visible light camera, such as a high resolution visible light camera, and an infrared camera. Some commercially available infrared cameras that are small enough to fit onto a handheld device don’t provide high enough resolution. Thus, the infrared camera may be paired with a high resolution visible light camera, and the image from the visible light camera may be overlaid with the infrared camera image. The resulting overlay image may then be processed to determine whether the spray administration was successful (on-target vs. off-target).

In any one of the embodiments described herein the system can also be used to actuate delivery of the medication when a user’s eyelid is open and/or when the eye is focused on a selected point and/or to avoid delivery when a user’s eye is closed or blinking.

The spray tracker system may use and/or execute one or more algorithm(s) to process the data collected by the spray tracker system via one or more cameras and/or sensors. The one or more algorithm(s) may process the data and produce one or more output(s). For example, images from the infrared camera and the visible light camera may be processed and/or compared by an algorithm to determine a location where the spray droplets were applied and/or to determine whether the spray was on-target and/or off-target. In one or more embodiments, the spray tracker system can determine the percentage of droplets that made it on- and/or off-target. The data from the cameras may include images from before, during, and after administration of the medication. The one or more algorithms may be executed by a processor.

The spray tracker system may further comprise an ophthalmic delivery device. The spray tracker system may be integrated into the body of an ophthalmic delivery device. In another embodiment, the spray tracker system is externally attached to an ophthalmic delivery device. For example, the spray tracker system may be attachable and/or adaptable to any commercially available dropper bottle and/or ophthalmic delivery device. The spray tracker system may be an aftermarket attachment and/or adapter. An ophthalmic delivery device may include, but is not limited to, a dropper bottle, spritzer, mister, and/or sprayer. The droplets may be standard droplets or micro-droplets. A micro-volume ophthalmic delivery device may be less than 30uL in total volume.

The spray tracker system and/or images may be used to determine if conditions of an application of medication are sufficient and/or otherwise require adjustment for increasing or decreasing a duration and/or amount of medication to be delivered. The spray tracker system may process the images from one or more sources to determine if the user has sufficiently or insufficiently medicated their eye surface. The system can be used and calibrated for optimal delivery and also for reduction of waste.

In any one or more of the embodiments described herein, the system is configured for use wherein the medication is a liquid formulation, such as a liquid suspension or aqueous solution and wherein the medication is delivered to the eye in the form of a droplet, microdroplet(s), a mist, or a spray.

In any one more of the embodiments described herein, the processor of the system is configured to determine an open or close state of an eye lid of the eye, an on or off target time for delivery of the medication, an ocular or periocular surface temperature, or combination thereof.

In any one more of the embodiments described herein, the processor utilizes an artificial intelligence algorithm for determination of one or more factors which control determination of successful delivery of medication to the eye and/or timing for delivery of medication.

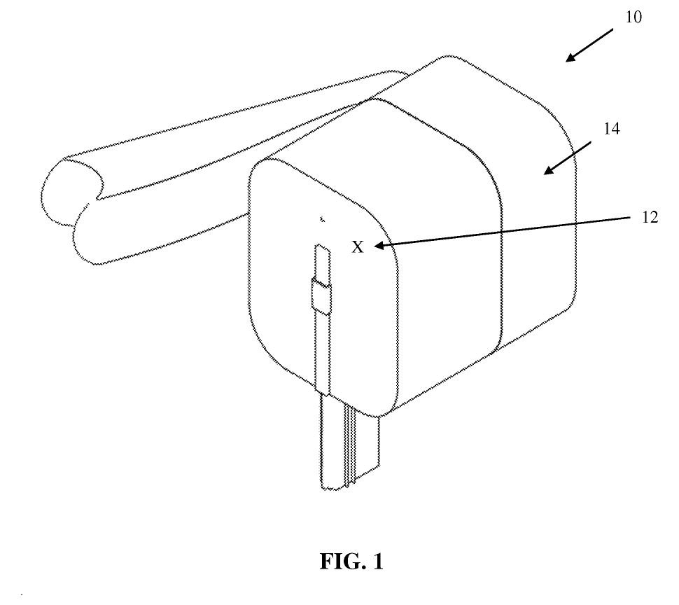

FIG. 1 illustrates a spray tracker system 10 according to one or more embodiments described herein. The spray tracker system 10 comprises one or more cameras and/or sensors 12. The spray tracker system 10 further comprises an ophthalmic medication delivery device 14. The spray tracker system 10 is integrated into the ophthalmic medication delivery device 14. The one or more cameras and/or sensors may include an infrared camera and a high resolution visible light camera to detect whether a spray was successfully applied to the surface of a user’s eye. The infrared camera may capture one or more images that may be used to determine a temperature change of the eye. For example, the infrared camera may capture one or more images before, during, and after the spray is applied to the eye. The high resolution visible light camera may capture one or more images that may be used to determine an alignment and/or openness/closeness of the eye before, during, and/or after the spray application. The spray tracker system 10 may further comprise a processor for processing the images captured from one or more cameras and/or sensors 12.

A method for delivery and/or tracking delivery of a medication to a user’s eye comprises capturing one or more first images of an eye surface of a user’s eye with one or more cameras 12, wherein at least one of the first images is taken before, during, and after a time when the medication is delivered to the eye surface. Then, capturing one or more second images of the user’s eye, wherein the one or more second images are taken during the time when the medication is delivered to the user’s eye, wherein the one or more second images are captured with the one or more cameras 12. And then, comparing the first one or more images and the one or more second images to determine a timing and/or a location of the user’s eye where the medication should be or was delivered.

The one or more first images of the user’s eye surface are captured with a first camera and the one or more second images are captured with an optional second camera. The first camera and optional second camera can be thermal cameras, optical cameras, or optionally a combination thereof.

The images captured are then compared by the processor to determine an open or close state of an eye lid of the eye, an on or off target time for delivery of medication, an ocular or periocular surface temperature or combination thereof and coordinating the information captured by the one or more cameras with the drug delivery device to independently deploy medication between the user’s blinks.

Although the present disclosure has been described with reference to preferred embodiments, workers skilled in the art will recognize that changes may be made in form and detail without departing from the spirit and scope of the disclosure.

1. A system for tracking delivery of medication to a user’s eye comprising:
one or more cameras for capturing one or more images of the user’s eye, before, during,
and after delivery of medication to the user’s eye.

2. The system of claim 1, wherein the one or more cameras are provided on the delivery device for delivery of the medication to the user’s eye and in operable connection therewith.

3. The system of claim 1, wherein the one or more cameras are provided with the delivery device for delivery of the medication to the user’s eye and are configured to track delivery of medication to the user’s eye from the delivery device without being integrated into the delivery device.

4. The system of claim 1, wherein the one or more cameras comprise a thermal camera, an optical camera, a multispectral camera, hyperspectral camera, or a combination thereof and wherein the one or more images comprise one or more optical images, thermal images or combinations thereof.

5. The system of claim 1, wherein the system comprises a single infrared camera to capture one or more images of the user’s eye before, during, and after delivery of medication to the user’s eye and a processor configured to compare one or more images captured before, during, or after delivery of medication to the user’s eye to determine if the medication was successfully applied to the user’s eye.

6. The system of claim 1, wherein the system comprises an infrared camera and a visible light camera to capture one or more images of the user’s eye before, during, and after delivery of medication to the user’s eye and a processor configured to compare one or more images captured from one or both of the infrared camera and visible light camera before, during, or after delivery of medication to the user’s eye to determine if the medication was successfully applied to the user’s eye.

7. The system of claim 1, wherein the one or more cameras are configured for capturing one or more first optical or thermal images of a surface of the user’s eye, before, during, and after delivery of medication.

8. The system of claim 1 further comprising a processor for comparing one or more images captures before, during, and after delivery of medication to the user’s eye to determine if the medication was successfully applied to the user’s eye.

9. The system of claim 1 wherein the medication is a liquid formulation.

10. The system of claim 1, wherein the medication is a liquid suspension or aqueous solution.

11. The system of claim 1, wherein the delivery device is configured to deliver the medication in the form of a droplet, a mist, or a spray.

12. The system of claim 1 and further comprising a processor configured to determine an open or close state of an eye lid of the eye, an on or off target time for delivery of medication, an ocular or periocular surface temperature or combination thereof.

13. The system of claim 11 wherein the processor utilizes an artificial intelligence algorithm for determination.

14. The system of claim 1 and further comprising one or more sensors for determining a position of user’s eye lid, a focal point of the user, a distance of the droplet dispersion, speed of the droplet dispersion or combinations thereof.

15. A method for tracking delivery of a medication to a user’s eye comprising:
capturing one or more first images of an eye surface of a user’s eye with one or more cameras, wherein at least one of the first images is taken before, during, and after a time when the medication is delivered to the eye surface;
capturing one or more second images of the user’s eye, wherein the one or more second images are taken during the time when the medication is delivered to the user’s eye, wherein the one or more second images are captured with the one or more cameras; and
comparing the first one or more images and the one or more second images to determine a timing and/or a location of the user’s eye where the medication should be or was delivered.

16. The method of claim 15 and further comprising providing the one or more cameras on a delivery device for the delivery of the medication to the user’s eye.

17. The method of claim 15 and capturing the one or more first images of the user’s eye surface with a first camera and capturing the one or more second images with a second camera wherein the first camera and second camera comprise a thermal camera, optical camera, or combination thereof.

18. The method of claim 15 and further comprising using the one or more first and one or more second images to determine an open or close state of an eye lid of the eye, an on or off target time for delivery of medication, an ocular or periocular surface temperature or combination thereof and coordinating the information captured by the one or more cameras with the drug delivery device to independently deploy medication between the user’s blinks.
